(19) United States
(12) Patent Application Publication
(10) Pub. No.: US 2011/0144496 A1
(43) Pub. Date: Jun. 16, 2011

(54) IMAGING METHOD FOR MICROCALCIFICATION IN TISSUE AND IMAGING METHOD FOR DIAGNOSING BREAST CANCER

(76) Inventors: Meng-Lin LI, Hsinchu (TW); Tsai-Chu HSIAO, Hsinchu (TW); Shin-Cheh CHEN, Hsinchu (TW); Yao-Yu CHENG, Kaohsiung City (TW); Po-Hsun WANG, Kaohsiung City (TW); Chih-Tai FAN, Taipei City (TW)

(21) Appl. No.: 12/891,401

(22) Filed: Sep. 27, 2010

(30) Foreign Application Priority Data
Dec. 15, 2009 (TW) 098142816

(51) Int. Cl. A61B 8/14

(52) U.S. Cl. 600/443

(57) ABSTRACT

An imaging method for microcalcification displays microcalcification distribution by acquiring and overlapping a photoacoustic image of microcalcification and an ultrasonic image of tissue. The image acquired by the present invention, in comparison to images acquired by ultrasonic and X-ray mammography, has advantages in no speckle noises, higher optical contrast, higher ultrasonic resolution, and so on. The present invention also has advantage in safety by adopting a light source having no ionizing radiation. An imaging method for diagnosing breast cancer is also herein disclosed.

### BACKGROUND OF THE INVENTION

1. Field of the Invention

The present invention relates to an imaging method for microcalcification in tissue and imaging method for diagnosing breast cancer, particularly to an imaging method for microcalcification in tissue and imaging method for diagnosing breast cancer by acquiring and superimposing a photoacoustic image of microcalcification and an ultrasonic image of tissue.

2. Description of the Prior Art

Microcalcification in breast has been one of the important indicators for early diagnosis for breast cancer and the location and distribution of microcalcification is also an important indicator for distinguishing malignant breast tumor. Therefore, detection of microcalcification in the breast is important for early diagnosis of breast tumor. X-ray mammogram is the most economical and effective screening tool among all and the only approved screening tool for breast cancer in many countries, including the United States, around the world. X-ray mammogram has been provided with high sensitivity for breast microcalcification and most microcalcification may be clearly observed in the X-ray film.

However, the X-ray mammogram may display microcalcification but not other subtle structures, such as mammary ducts. The result of X-ray mammogram for a patient can not be directly interpreted by radiologists and a further ultrasound test is needed to be performed by doctors. However, due to speckle noises present in the ultrasound image and relative low contrast between the breast tissue and microcalcification, the sensitivity of the microcalcification image is less than 30%. Therefore, it has been a huge challenge for all clinical practitioners to find out the position of suspected tumor and microcalcification found in X-ray mammogram using an ultrasound system.

In addition, photoacoustic imaging has been used for breast cancer screening. Photoacoustic imaging or photoacoustic tomography (PAT) is performed by using laser to induce ultrasound and has advantages in high contrast of optical imaging as well as the high penetration depth and high resolution of ultrasound. The photoacoustic imaging may choose various wavelengths for the light source based on the absorption spectrum feature to obtain images for various tissues. However, the photoacoustic imaging used for screening the breast cancer is mainly focused on blood objects, such as angiogenesis and hemorrhagic infiltration.

To sum up, it is now a current goal to develop an effective screening method for breast microcalcification.

### SUMMARY OF THE INVENTION

The present invention is directed to an imaging method for microcalcification which displays microcalcification distribution by acquiring and overlapping a photoacoustic image of microcalcification and an ultrasonic image of tissue. The image acquired by the present invention, in comparison to images acquired by ultrasonic and X-ray mammography, has advantages in no speckle noises, higher optical contrast, higher ultrasonic resolution, and so on.

In one embodiment, an imaging method for microcalcification in tissue including emitting a first ultrasound to a tissue; receiving an echo wave of the first ultrasound and forming a first ultrasound image of the tissue; emitting a first light to the tissue to induce a first photoacoustic ultrasound; receiving the first photoacoustic ultrasound and forming a first photoacoustic image of a microcalcification; and superimposing the first ultrasound image and the first photoacoustic image to form a first superimposed image for illustrating the microcalcification distributed within the tissue.

The present invention is also directed to an imaging method for diagnosing breast cancer, which displays microcalcification in the breast to analyze the stage and phase of the breast cancer. The present invention adopts a light source having no ionizing radiation and thus has an advantage in safety.

In another embodiment, an imaging method for diagnosing breast cancer includes emitting a first ultrasound to a breast tissue; receiving an echo wave of the first ultrasound and forming a first ultrasound image of the breast tissue; emitting a first light to the tissue to induce a first photoacoustic ultrasound; receiving the first photoacoustic ultrasound and forming a first photoacoustic image of a microcalcification; and superimposing the first ultrasound image and the first photoacoustic image to form a first superimposed image for illustrating the microcalcification distributed within the breast tissue and diagnosing the phase and stage of breast cancer.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The patent or application file contains at least one drawing executed in color. Copies of this patent application publication with color drawing(s) will be provided by the Office upon request and payment of the necessary fee.

The foregoing aspects and many of the accompanying advantages of this invention will become more readily appreciated as the same becomes better understood by reference to the following detailed descriptions, when taken in conjunction with the accompanying drawings, wherein:

FIG. 1 is a flow chart illustrating an imaging method for microcalcification according to an embodiment of the present invention;

FIG. 2 is a schematic diagram illustrating an integrated system of photoacoustic imaging and ultrasound imaging according to an embodiment of the present invention;

FIGS. 3a-3b are diagrams illustrating the preferred infrared wavelength according to one embodiment of the present invention;

FIG. 4 is a flow chart illustrating a diagnostic method for breast cancer according to one embodiment of the present invention;

FIG. 5 is an ultrasound image of the mock tissue according to an embodiment of the present invention;

FIG. 6 is a photoacoustic ultrasound image according to an embodiment of the present invention; and

FIG. 7 is an imaging result illustrating the microcalcification in the tissue according to one embodiment of the present invention.

### DESCRIPTION OF THE PREFERRED EMBODIMENT

The present invention is directed to a superimposed image generated from a photoacoustic image of microcalcification on an ultrasound image of the tissue. The superimposed image is used for illustrating microcalcification distributed within the tissue.

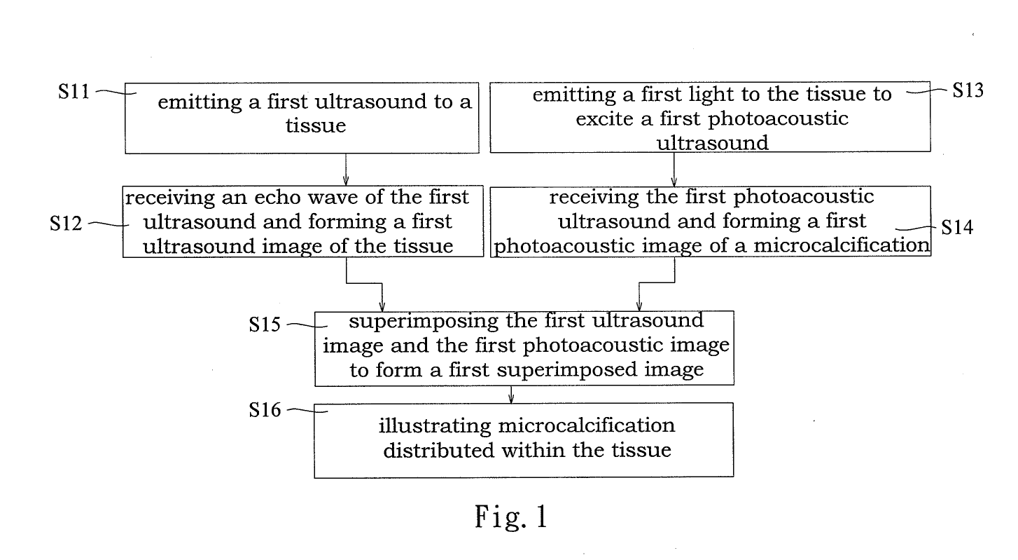

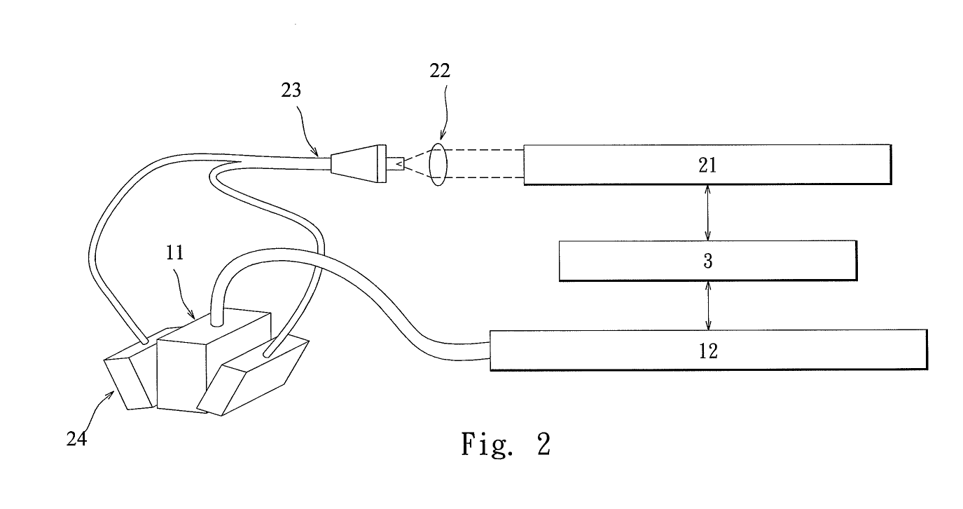

Refer to FIG. 1 and FIG. 2, where FIG. 1 is a flow chart illustrating an imaging method for microcalcification according to an embodiment of the present invention, and FIG. 2 is a schematic diagram illustrating an integrated system of photoacoustic imaging and ultrasound imaging according to an embodiment of the present invention.

First, Steps S11 and S12 are performed to obtain an ultrasound image of ROI (Region of Interest) tissue. Step S11 includes emitting a first ultrasound to a tissue. The ultrasound may be generated with an ultrasound array transducer 11. Short electrical pulses generated with the ultrasound array transducer 11 result in the ultrasound at the desired frequency. The ultrasound array transducer 11 may be integrated with a medical ultrasound array imaging system 12. The ultrasound array transducer 11 having desired frequency may be chosen based on resolution and penetrating depth; for example, a transducer having a middle frequency such as 10 MHz may be chosen for 5 cm depth and 300 μm resolution.

Step S12 includes receiving an echo wave of the first ultrasound and forming a first ultrasound image of the ROI tissue. The sound wave is effectively transmitted into the ROI tissue with the assistance of the ultrasound array transducer 11 and partially reflected from the layers between different tissues where density changes occur in tissue, e.g. the interface between tumor cells and normal cells within the tissue or the interface between the tissue and the cysts. The partially reflected echo wave would return to and oscillate the ultrasound array transducer 11 and be absorbed by the ultrasound array transducer 11. The ultrasound array transducer 11 then converts the oscillation into electrical impulse signal, which is received, amplified, modulated and displayed by the ultrasound array imaging system 12.

Here, the common display modes adopted in medical field include A mode (Amplitude Mode), B mode (Brightness Mode), M mode (Motion Mode), D mode (Doppler Mode) and so on.

In addition, the first ultrasound image may be classified into a 2D image, a 3D image or a Doppler image based on the imaging theory of ultrasound. The 2D ultrasound image is a planar sectional view displaying internal structures within the tissue for observing the shape and size of structures. The 3D ultrasound image, processed by a computer, is formed by recombining a series of adjacent 2D ultrasound images with enhanced contrast and displaying a three-dimensional ultrasound image on the screen. For real-time 3D ultrasound imaging, a two-dimensional array ultrasound transducer is adopted for acquiring images in a 3-dimensional manner to display 3D ultrasound image on the screen in a real-time manner. For Doppler ultrasound imaging, Doppler Effect is applied for resolving the blood flow rate and vessel distribution.

Step S13 and Step S14 are next performed to obtain a photoacoustic image for microcalcification. Step S13 includes emitting a first light, preferably a laser beam, to the ROI tissue to induce a first photoacoustic ultrasound. As illustrated in FIG. 2, the laser beam emitted by the impulse laser system 21 is guided to the linear optical guide array 24 through the lens 22 and beam splitter 23 to scan the ROI tissue for obtaining photoacoustic images. For photoacoustic imaging mode, the impulse laser system 21 and ultrasound array imaging system 12 may be synchronized by the laser control unit 3. Therefore, the laser beam emitted from the impulse laser system 21 may be triggered either by the ultrasound array imaging system 12 or by the impulse laser system 21 itself for photoacoustic imaging.

As above-mentioned, the light for photoacoustic imaging is a laser beam, preferably. For example, the laser beam having tunable wavelength may be generated by an optical parametric oscillator driven by a Nd:YAG impulse laser. For example, the output laser may have 3˜20 ns in impulse width, 10 Hz˜KHz in pulse repetition frequency (PRF) and tunable 410˜4000 nm in wavelength. The weak focus of laser energy on the target is achieved by using dark-field and is confocal with the elevational direction of the ultrasound array transducer 11. Here, the dark field is used for preventing interference caused by strong photon absorption of surface, and the confocal configuration may enhance SNR of images.

In addition, partial laser energy may be coupled into the optical fiber used for monitoring laser energy. The output energy guided by the optical fiber is monitored by LED and used for post processing for eliminating instable output energy of the laser. The above-mentioned configuration is not thus limited, however. Any other configuration that adopts other laser radiation to the target and monitors laser energy may be used as long as the aforesaid function is achieved. Here, the energy density of the impulse laser exposed to the tissue surface should be less than the allowable maximum defined by ANSI.

Microcalcification has now been related to some acute or chronic diseases, such as acute inflammation or tumor. Microcalcification may be distributed in tissues, for example, without limitation to breast, blood vessel, lung, thyroid or kidney.

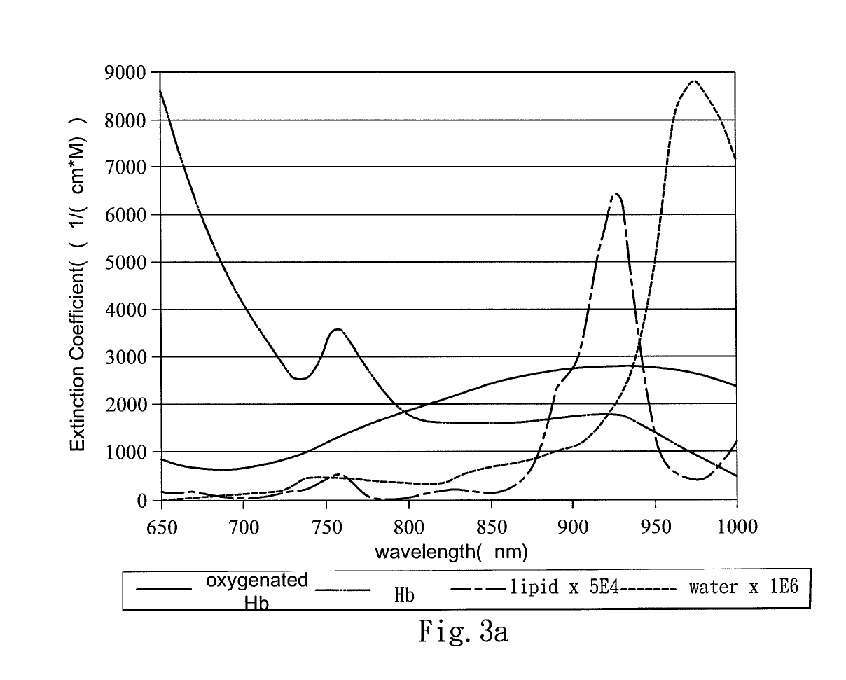

Microcalcification imaging is preferably achieved by using near infrared light, having 700 nm˜1200 nm wavelength. Referring to FIG. 3a, cited from T. J. Brukilacchio, Ph.D. Thesis 2003, near infrared light has deeper penetration depth in tissue and is less absorbed by the blood components such as hemoglobin (Hb), oxygenated Hb, lipid and water. To be specific, the infrared light having <700 nm wavelength is greatly absorbed by the non-oxygenated Hb, and the infrared light having >900 nm is greatly absorbed by the lipid; therefore, the 700-850 nm forms the preferred optical window for breast tissue transmission. In addition, referring to FIG. 3b, within the range of 700-850 nm, the optical absorbance or photoacoustic signal for microcalcification is greater in comparison to blood, lipid or gland tissue. Therefore, the reading of a microcalcification image would be less influenced by the blood, lipid, or gland signal.

In addition, the microcalcification in the breast has been related to tumor malignancy in literature. The major composition of mammary microcalcification includes calcium oxalate, calcium hydroxyapatite, calcium carbonate hydroxyapatite or the combinations thereof. Calcium oxalate is mainly present in non-invasive tumors and calcium hydroxyapatite is mainly found in invasive tumors. Particularly, the more phosphate is replaced by carbonate in calcium carbonate hydroxyapatite, the higher possibility the tumors are non-invasive. Due to absorbance difference of calcium oxalate, calcium hydroxyapatite and calcium carbonate hydroxyapatite in 3200 nm to 3600 nm, light source having wavelength in 3200 nm to 3600 nm may used for photoacoustic imaging of microcalcification characteristic absorbance spectrum so as to qualitatively analyze microcalcification composition. The obtained result may further be used for risk determination of ductal carcinoma in situ and an objective basis for subsequent adoption of positive treatment or conservative observation.

Step S14 includes receiving the first photoacoustic ultrasound and forming a first photoacoustic image of the microcalcification. The photoacoustic and ultrasound integrated system may perform simultaneous capture for multi-array channel signals. The signals are pre-amplified and converted to digital signals by A/D converters. Beamforming of the ultrasound receive beamformer and dynamic focusing are then performed to form photoacoustic imaging. The above processing flow is the same as the signal processing system of receiving end of present ultrasound system. However, due to relative much faster speed of light comparing to the speed of sound, the traveling time of laser beam in the tissue may be neglected and only the time of photoacoustic echo needs to be considered when calculating imaging depth.

It is noted that the photoacoustic and ultrasound integrated system of the present invention may switch between the photoacoustic mode and ultrasound imaging mode and may sequentially and/or simultaneously capturing and displaying photoacoustic and ultrasound images for verifying the relative position of microcalcification.

Therefore, the formation of ultrasound and photoacoustic images, in terms of first ultrasound image and first photoacoustic image has no sequential limit.

In addition, the present invention may be used for forming consecutive dynamic images obtained by similar steps as above-mentioned, including emitting a second ultrasound to the tissue; receiving an echo wave of the second ultrasound and forming a second ultrasound image of the tissue; emitting a second light to the tissue to induce a second photoacoustic ultrasound; receiving the second photoacoustic ultrasound and forming a second photoacoustic image of a microcalcification; and superimposing the second ultrasound image and the second photoacoustic image to form a second superimposed image. Preferably, a frame of the first photoacoustic image is formed between a frame of the first ultrasound and a frame of the second ultrasound. Since the photoacoustic imaging frame is sandwiched between two sequential B-mode pulse echo frames of ultrasound, the system of the present invention has no difference from present medical ultrasound array system in the way it is operated and remains using the operating method of conventional ultrasound system without additional scanning time for adding a photoacoustic image of the same resolution as the ultrasound image.

As fore-mentioned, the first ultrasound image may be 2D ultrasound images or 3D ultrasound images. The scanning method of the first photoacoustic image which may be determined by the positional configuration of the ultrasound array transducer 11 and the linear optical guide array 24 includes backward mode, forward mode and tomography mode. The first photoacoustic image may be 2D ultrasound images or 3D ultrasound images as above-mentioned.

Finally, Step S15 includes superimposing the first ultrasound image and the first photoacoustic image so as to form a first superimposed image for illustrating microcalcification distributed within the tissue (Step S16).

In one embodiment, the present invention provides a 2D projection image similar to the X ray photography. As fore-mentioned, X ray photography is the major detecting method at present and a 2D image is obtained by projecting the test object along a projection line. The 3D first photoacoustic image or the first superimposed image may be projected to obtain a 2D projection image so that the clinical practitioners may obtain projection result similar to the X ray photography to replace and/or to compare with standard mammary X ray photography.

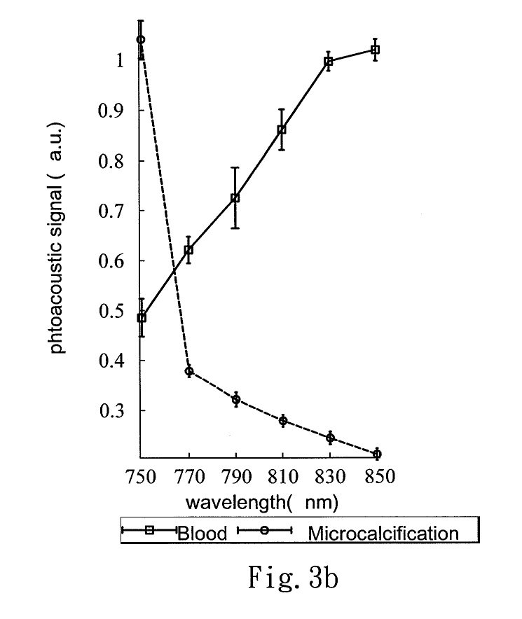

As mentioned previously, microcalcification in breast is known as one of the important indicators for early diagnosis of breast cancer. FIG. 4 is a flow chart illustrating a diagnostic method for breast cancer. Referring to FIG. 4, a diagnostic method for breast cancer includes emitting a first ultrasound to a breast tissue (Step S21); receiving an echo wave of the first ultrasound and forming a first ultrasound image of the breast tissue (Step S22); emitting a first light, preferably a laser beam to the breast tissue to induce a first photoacoustic ultrasound (Step S23); receiving the first photoacoustic ultrasound and forming a first photoacoustic image of the microcalcification (Step S24); and superimposing the first ultrasound image and the first photoacoustic image to form a first superimposed image (Step 25); illustrating microcalcification distributed within the breast tissue and determining the stage and phase of the breast cancer (Step S26). Here, the Steps S21-S25 are similar to the above-mentioned steps and are not detailed.

For determining the stage and phase of the breast cancer (Step S26), the distribution, density, shape and composition of the microcalcification may be taken into consideration. For example, the more microcalcification distributed within a certain part, the higher diagnostic probability of malignant tumor; and microcalcification of irregular shapes, such as linear shape, radial shape, and fork shape may lead to malignant tumor. Therefore, clinical practitioners may easily find the location of microcalcification in the breast ultrasound image by using the present invention for the stage and phase determination of the ductal carcinoma in situ and the objective basis for subsequent adoption of positive treatment or conservative observation.

The following description describes a specific embodiment of the present invention used for detecting the microcalcification in a biological sample mock. The mock composition includes gelatin (used for simulating the biological tissue), cellulose (used for simulating the speckle noise in the ultrasound image), Intralipid (used for simulating the light scattering within the tissue) and HA particles (used for simulating the microcalcification associated with malignant breast tumor.

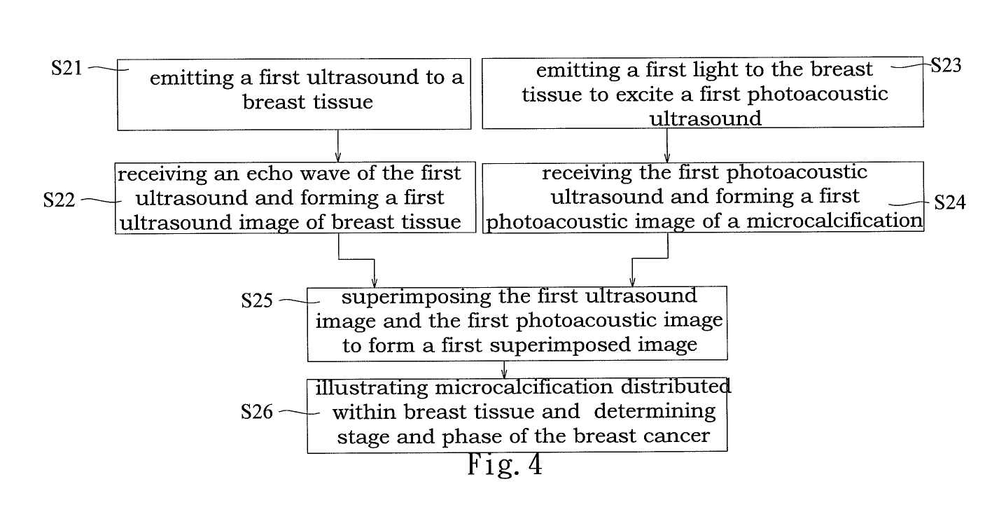

Refer to FIG. 5, which is an ultrasound image of the mock tissue according to an embodiment of the present invention, where the image mode is B mode, the dynamic range is 35 db, and the mock is fixed in the water. As illustrated, the upper dark region having no reflection illustrates the water where the mock is placed in, and the surface of the mock is illustrated at 12 mm in depth. There are a lot of speckle noises in the mock image masking the signal of microcalcification. Therefore, due to insufficient contrast in the image, the location where the microcalcification is present is very difficult to tell.

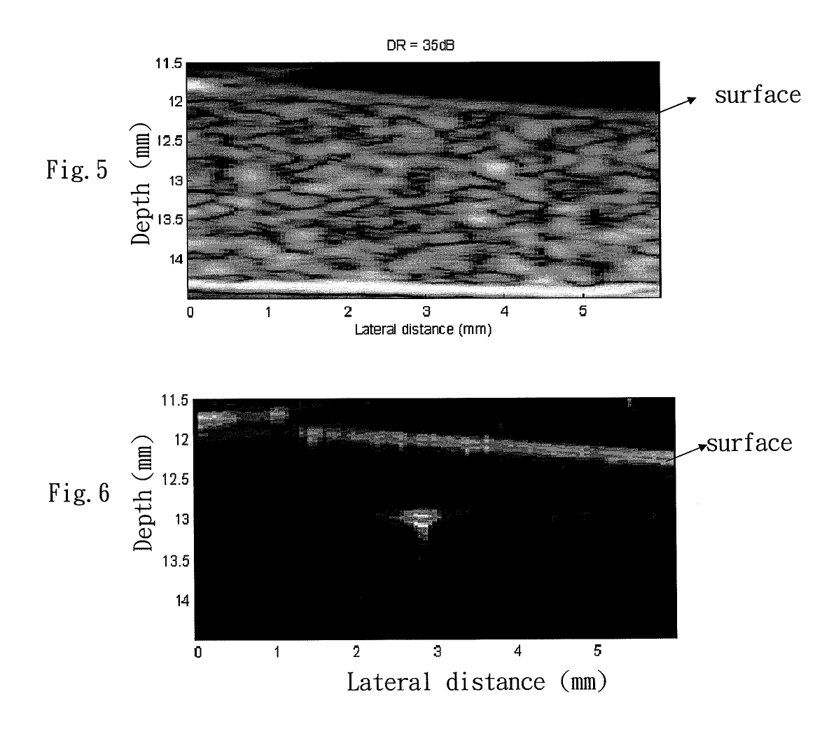

Refer to FIG. 6, which is a photoacoustic ultrasound image according to an embodiment of the present invention. A photoacoustic image mode, setting of which is 800 nm in wavelength and B mode, is adopted for scanning in the same scanning area. The RF (radio frequency) data obtained from scanning is then processed with envelope detection to obtain the illustrated result, where the brightness of the image is expressed in linear scale. As illustrated in FIG. 6, the surface is illustrated at 12 mm in depth, and an obvious bright spot, i.e. the microcalcification, is illustrated at 13 mm in depth.

Due to weak absorption to 800 nm infrared, both the peripheral tissue around the microcalcification and water above the mock have weak photoacoustic signal and display dark in the image; therefore, it shows strong image contrast between the microcalcification and the background in the photoacoustic image.

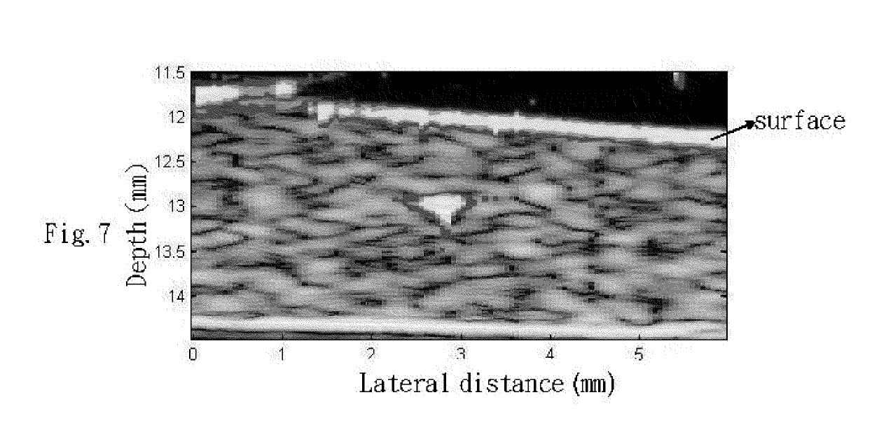

Refer to FIG. 7, which illustrates an imaging result of the microcalcification in the tissue according to one embodiment of the present invention. FIG. 7 illustrates the superimposed result of the ultrasound image illustrated in FIG. 5 and the photoacoustic image illustrated in FIG. 6, wherein the ultrasound image is displayed in grayscale and the photoacoustic image is display in red color (pseudo-color). The completely overlapped result of these two surface signals demonstrates FIG. 5 and FIG. 6 illustrate an identical cross section. The superimposed image illustrates a clearer result by showing the structural distribution of the breast mock of the ultrasound image and the presence of the microcalcification illustrated in the photoacoustic image.

To sum up, by acquiring and overlapping a photoacoustic image of microcalcification and an ultrasonic image of tissue, the present invention may display microcalcification distribution to further analyze the phase and stage of breast cancer. The image acquired by the present invention, in comparison to images acquired by ultrasonic and X-ray mammography, has advantages in no speckle noises, higher optical contrast, higher ultrasonic resolution, and so on. The present invention also has an advantage in safety by adopting a light source having no ionizing radiation.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An imaging method for microcalcification in tissue, comprising:
emitting a first ultrasound to a tissue;
receiving an echo wave of the first ultrasound and forming a first ultrasound image of the tissue;
emitting a first light to the tissue to induce a first photoacoustic ultrasound;
receiving the first photoacoustic ultrasound and forming a first photoacoustic image of a microcalcification; and
superimposing the first ultrasound image and the first photoacoustic image to form a first superimposed image for illustrating the microcalcification distributed within the tissue.

2. The imaging method as claimed in claim 1, wherein the light is a laser beam.

3. The imaging method as claimed in claim 2, wherein the laser beam has a wavelength in the range of 3200 nm to 3600 nm.

4. The imaging method as claimed in claim 2, wherein the laser beam has a wavelength in the range of 700 nm to 1200 nm.

5. The imaging method as claimed in claim 2, wherein the laser beam has a wavelength in the range of 700 nm to 850 nm.

6. The imaging method as claimed in claim 1, wherein the first ultrasound image comprises a 2D ultrasound image, a 3D ultrasound image or a Doppler ultrasound image.

7. The imaging method as claimed in claim 1, wherein the first photoacoustic image comprises a 2D ultrasound image or a 3D ultrasound image.

8. The imaging method as claimed in claim 1, wherein the first photoacoustic image is formed with backward mode, forward mode or tomography mode.

9. The imaging method as claimed in claim 1, wherein the microcalcification comprises calcium oxalate, calcium hydroxyapatite, calcium carbonate hydroxyapatite or the combinations thereof.

10. The imaging method as claimed in claim 1, wherein the tissue comprises breast, blood vessel, lung, thyroid or kidney.

11. The imaging method as claimed in claim 1, further comprising:
projecting the first photoacoustic image or the first superimposed image to obtain a 2D projecting image, wherein the first photoacoustic image or the first superimposed image is 3-Dimensional.

12. The imaging method as claimed in claim 1, wherein the first photoacoustic image is obtained along a projection line of an X-ray photography.

13. The imaging method as claimed in claim 1 further comprising:
emitting a second ultrasound to the tissue;
receiving an echo wave of the second ultrasound and forming a second ultrasound image of the tissue;
emitting a second light to the tissue to induce a second photoacoustic ultrasound;
receiving the second photoacoustic ultrasound and forming a second photoacoustic image of a microcalcification; and
superimposing the second ultrasound image and the second photoacoustic image to form a second superimposed image, wherein a frame of the first photoacoustic image is formed between a frame of the first ultrasound and a frame of the second ultrasound.

14. An imaging method for diagnosing breast cancer, comprising:
emitting a first ultrasound to a breast tissue;
receiving an echo wave of the first ultrasound and forming a first ultrasound image of the breast tissue;
emitting a first light to the breast tissue to induce a first photoacoustic ultrasound;
receiving the first photoacoustic ultrasound and forming a first photoacoustic image of a microcalcification; and
superimposing the first ultrasound image and the first photoacoustic image to form a first superimposed image for illustrating the microcalcification distributed within the breast tissue and diagnosing the phase and stage of breast cancer.

15. The imaging method as claimed in claim 14, wherein the light is a laser beam.

16. The imaging method as claimed in claim 15, wherein the laser beam has a wavelength in the range of 3200 nm to 3600 nm.

17. The imaging method as claimed in claim 15, wherein the laser beam has a wavelength in the range of 700 nm to 1200 nm.

18. The imaging method as claimed in claim 15, wherein the laser beam has a wavelength in the range of 700 nm to 850 nm.

19. The imaging method as claimed in claim 14, wherein the first ultrasound image comprises a 2D ultrasound image, a 3D ultrasound image or a Doppler ultrasound image.

20. The imaging method as claimed in claim 14, wherein the first photoacoustic image comprises a 2D ultrasound image or a 3D ultrasound image.

21. The imaging method as claimed in claim 14, wherein the first photoacoustic image is formed with backward mode, forward mode or tomography mode.

22. The imaging method as claimed in claim 14, wherein the microcalcification comprises calcium oxalate, calcium hydroxyapatite, calcium carbonate hydroxyapatite or the combinations thereof.

23. The imaging method as claimed in claim 14, further comprising:
projecting the first photoacoustic image or the first superimposed image to obtain a 2D projecting image, wherein the first photoacoustic image or the first superimposed image is 3-Dimensional.

24. The imaging method as claimed in claim 14, wherein the first photoacoustic image is obtained along a projection line of an X-ray photography.

25. The imaging method as claimed in claim 14, wherein the phase and stage of breast cancer is diagnosed via density distribution, shape or composition of the microcalcification.

26. The imaging method as claimed in claim 14 further comprising:
emitting a second ultrasound to the breast tissue;
receiving an echo wave of the second ultrasound and forming a second ultrasound image of the breast tissue;
emitting a second light to the breast tissue to induce a second photoacoustic ultrasound;
receiving the second photoacoustic ultrasound and forming a second photoacoustic image of a microcalcification; and
superimposing the second ultrasound image and the second photoacoustic image to form a second superimposed image, wherein a frame of the first photoacoustic image is formed between a frame of the first ultrasound and a frame of the second ultrasound.
